(19) United States
(12) Patent Application Publication
(10) Pub. No.: US 2012/0253172 A1
(43) Pub. Date: Oct. 4, 2012

(54) Radiation therapy system with high frequency shielding

(76) Inventors: Wilfried Loeffler, Erlangen (DE); Bela Vajko, Sagamore Hills, OH (US)

(21) Appl. No.: 13/432,112

(22) Filed: Mar. 28, 2012

(30) Foreign Application Priority Data
Mar. 31, 2011 (DE) 102011006582.2

(51) Int. Cl. A61N 5/10; A61B 5/055

(52) U.S. Cl. 600/411

(57) ABSTRACT

A radiation therapy system is proposed. The system has a magnetic resonance imaging device with a first and second magnetic coil units distanced from one another by a gap. The coil units interact to generate a magnetic field propagating along a longitudinal axis. The system has a linear accelerator arranged at a radial distance from the longitudinal axis in the gap or in the radial extension of the gap. The system also has a high frequency shielding arrangement with a first and second high frequency shielding cabins arranged adjacent to the first and second magnetic coil units respectively and high frequency sealed connected to one another with a tubular high frequency shielding unit arranged along the longitudinal axis within the first and second magnetic coil units. A high frequency shielding concept is independent of an embodiment of the magnetic system of the imaging device and is universally applicable.

### CROSS REFERENCE TO RELATED APPLICATIONS

This application claims priority of German application No. 10 2011 006 582.2 filed Mar. 31, 2011, which is incorporated by reference herein in its entirety.

### FIELD OF INVENTION

The invention relates to a radiation therapy system which includes a magnetic resonance imaging device, a linear accelerator and a high frequency shielding arrangement.

### BACKGROUND OF INVENTION

For improved and more accurate radiation therapy, the advantages of a medical linear accelerator (linac), which generates high-energy x-ray radiation to treat tumors, and the advantages of a medical magnetic resonance imaging (MR) to pictorially represent tumors have for some time been combined in a combination device. US 2011/0012593 A1 discloses such a combination device with a split magnetic system of the MR components, whereby the linac is positioned in a gap between a first and a second magnetic coil system.

In order to generate artifact-free MR images, a shielding against external high frequency fields is an important prerequisite. Shield attenuations of 100 dB to 125 dB are needed here in order to shield radio transmitters for instance. With linac-MR combination devices, it is necessary, for simultaneous operation of both systems, to shield the high frequency interference radiation generated by different components of the linac from the patient and the high frequency receiving antennae of the MR device in addition to the external high frequency interference fields.

In M. Lamey et al., “Radio frequency shielding for a linac-MRI system”, Phys. Med. Biol. 55 (2010), pages 995-1006, a linac-MR arrangement is surrounded by an external high frequency shield and the microwave generation of the linac is arranged outside of the HF shield. The high frequency energy is brought into the interior of the HR shielding by means of a hollow conductor. Further components needed for a linac operation, such as for instance the electron injector, remain inside the shield and must be be additionally shielded if necessary.

### SUMMARY OF INVENTION

It is the object of the invention to specify a linac-MR radiation therapy system including a split MR magnetic system with an improved high frequency shielding.

According to the invention, the set object is achieved with the radiation therapy system of the independent claims.

The invention claims a radiation therapy system with a magnetic resonance imaging device having a first and a second magnetic coil unit, which are distanced from on another by means of a gap, whereby the first and second magnetic coil unit interact such that they generate a magnetic field which propagates at least partially along a longitudinal axis. The system further includes a linear accelerator, which is arranged at a radial distance from the longitudinal axis in the gap or in the radial extension of the gap, and a high frequency shielding arrangement. The shielding arrangement includes a first and a second high frequency shielding cabin arranged adjacent to the first magnetic coil unit and to the second magnetic coil unit in each instance, which are connected to one another in a high frequency sealed fashion with a tubular high frequency shielding unit arranged along the longitudinal axis within the first and second magnetic coil unit. The invention is advantageous in that a high frequency shielding concept is independent of the embodiment of the magnetic system of the magnetic resonance imaging and can thus be used universally. The linear accelerator is completely outside of the high frequency shielding arrangement, an additional shielding of linac components is therefore not necessary.

The invention also claims a radiation therapy system with a magnetic resonance imaging device having a first and a second magnetic coil unit which are distanced from one another by means of a gap, whereby the first and second magnetic coil unit interact such that they generate a magnetic field which propagates at least partially along a longitudinal axis. The radiation therapy system also includes a linear accelerator, which is arranged at a radial distance from the longitudinal axis in the gap or in the radial extension of the gap, as well as a high frequency shielding arrangement with a first high frequency shielding cabin arranged adjacent to the first magnetic coil unit and connected thereto in a high frequency sealed fashion, a second high frequency shielding cabin arranged adjacent to the second magnetic coil unit and connected thereto in a high frequency sealed fashion and a tubular high frequency shielding unit which bridges the gap and is arranged along the longitudinal axis, said high frequency shielding unit being connected to the first and second magnetic coil unit in a high frequency sealed fashion. The invention is advantageous in that the high frequency shielding of the magnetic resonance imaging can take place in a simple and precise fashion.

In a development of the invention, the first and/or second high frequency shielding cabin can be embodied such that people can remain inside.

In a further embodiment, a first door can be embodied in the first high frequency shielding cabin.

Furthermore, a second door can be embodied in the second high frequency shielding cabin.

In a development, the tubular high frequency shielding unit can be embodied such that an energy-rich x-ray radiation of the linear accelerator penetrates the high frequency shielding unit.

### BRIEF DESCRIPTION OF THE DRAWINGS

Further details and advantages of the invention are apparent from the subsequent explanations of several exemplary embodiments with the aid of schematic drawings, in which;

FIG. 1: show a block diagram of a linac MR arrangement according to the prior art,

FIG. 2: shows a block diagram of a radiation therapy system with a high frequency shielding arrangement and

FIG. 3: shows a block diagram of a radiation therapy system with a further high frequency shielding arrangement.

### DETAILED DESCRIPTION OF INVENTION

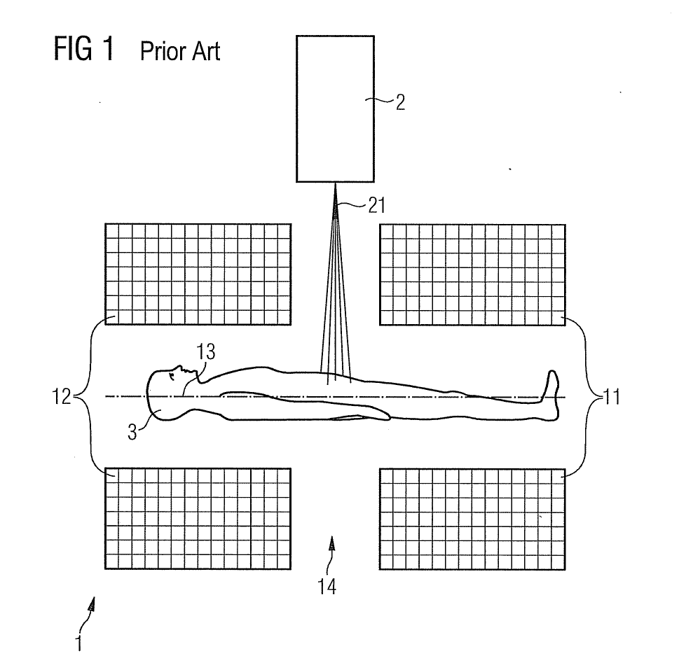

FIG. 1 shows a block diagram of a radiation therapy system having a linac MR arrangement according to US 2011/00112593 A1. The present invention can be combined with this arrangement. In this regard, reference is made to the application US 2011/00112593 A1, the contents of which is herewith included in this application. Parts of a magnetic resonance imaging device 1 are visible in a cross-section, namely the split magnetic coil system having a first magnetic coil unit 11 and a second magnetic coil unit 12. The two magnetic coil units 11, 12 are distanced from one another by means of a gap 14. Energy-rich x-ray radiation 21 of a linear accelerator 2 can be directed at a patient 3 through the gap 14. The two magnetic coil units 11 and 12 interact such that a magnetic field is generated which propagates at least partially along a longitudinal axis 13 which runs through the isocenter of the two magnetic coil units 11 and 12.

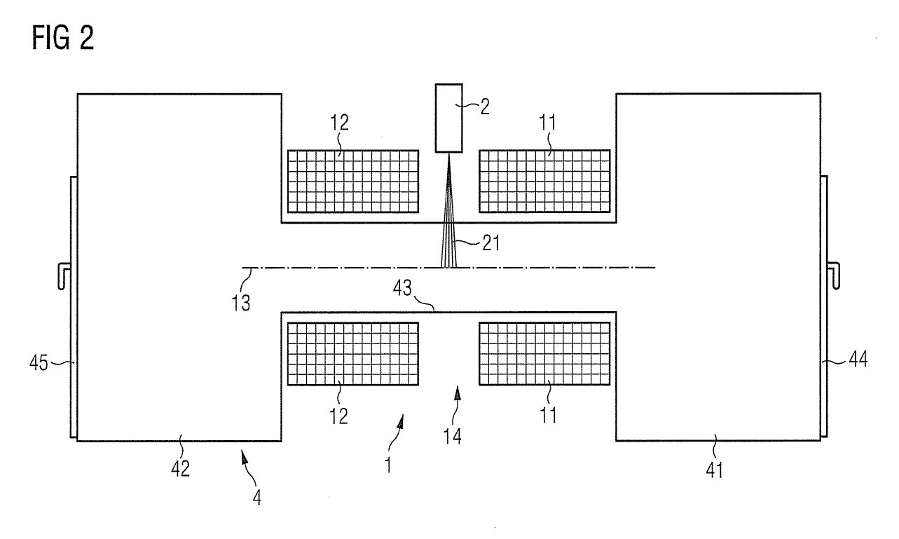

FIG. 2 shows a block diagram of a radiation therapy system having a first inventive embodiment of a high frequency shielding. Parts of a magnetic resonance imaging device 1 with a split magnetic coil system having a first magnetic coil unit 11 and a second magnetic coil unit 12 are shown cross-sectionally. The two magnetic coil units 11, 12 are distanced from one another by means of a gap 14. Energy-rich x-ray radiation 21 of a linear accelerator 2 can be directed at a patient 3 through the gap 14. The two magnetic coil units 11 and 12 interact such that a magnetic field is generated, which propagates at least partially along a longitudinal axis 13 which proceeds through the isocenter of the two magnetic coil units 11 and 12.

A high frequency shielding arrangement 4 is used in accordance with the invention in order to shield against high frequency interference radiation of the linear accelerator 2 and the microwave high frequency source needed for its operation as well as external high frequency interference fields. The high frequency shielding arrangement 4 includes a first high frequency shielding cabin 41, which is arranged on the side of the first magnetic coil unit 11 which faces away from the gap 14, as well as a second high frequency shielding cabin 42, which is arranged on the side of the second magnetic coil unit 12 which faces away from the gap 14. The cabins 41 and 42 are preferably embodied large enough for people to remain inside. The first high frequency shielding cabin 41 comprises a first door 44, the second high frequency shielding cabin 42 can comprise a second door 45 in order if necessary to provide operating personnel with access from both sides.

The two cabins 41 and 42 are connected to one another by means of a tubular high frequency shielding unit 43. The shielding unit 43 lies inside the first and second magnetic coil unit 11, 12 and forms a tube, into which a patient can be moved. The connections of the shielding unit 43 to the two cabins 41 and 42 must be embodied in a high frequency-tight fashion. The first and the second high frequency shielding cabins 41, 42 and the tubular high frequency shielding unit 43 can be made of copper sheeting and fowl a Faraday cage.

The tubular shielding unit 43 has a diameter of this type such that a patient and the couch and if necessary further MR imaging components such as gradient and HF coil for the treatment can be accommodated.

Tests and simulations show that an irradiation of the tubular shielding unit 43 with energy-rich x-rays of a linac is possible without any problem.

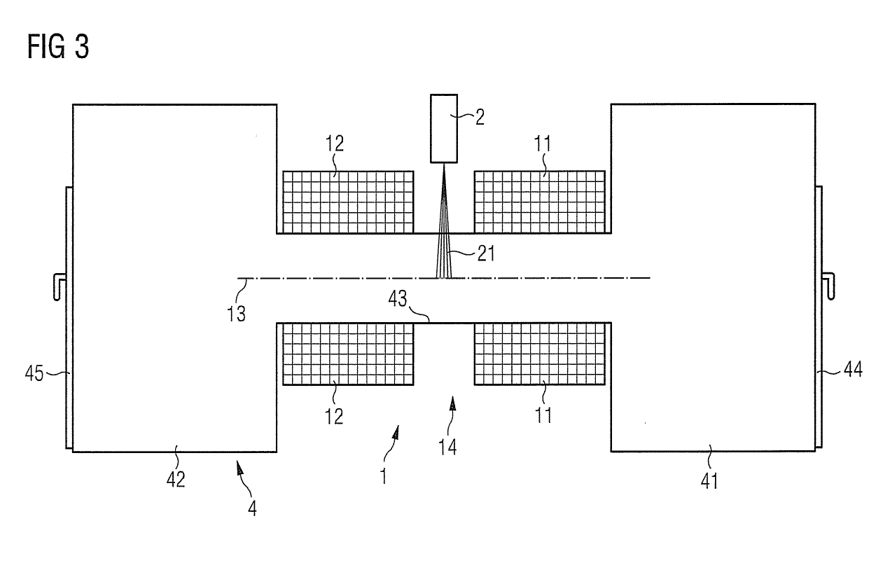

FIG. 3 shows a block diagram of a radiation therapy system having a second inventive embodiment of a high frequency shielding. Evident in the cross-section are parts of a magnetic resonance imaging device 1 with a split magnetic coil system having a first magnetic coil unit 11 and a second magnetic coil unit 12. The two magnetic coil units 11, 12 are distanced from one another by means of a gap 14. Energy-rich x-rays 21 of a linear accelerator 2 can be directed at a patient 3 through the gap 14. The two magnetic coil units 11 and 12 interact such that a magnetic field is generated which propagates at least partially along a longitudinal axis 13 which proceeds through the isocenter of the two magnetic coil units 11 and 12.

A high frequency shielding arrangement 4 is used to shield a high frequency interference radiation of the linear accelerator 2 and external high frequency interference fields. The high frequency shielding arrangement 4 includes a first high frequency shielding cabin 41, which is arranged on the side of the first magnetic coil unit 11 which faces away from the gap 14, as well as a second high frequency shielding cabin 42, which is arranged on the side of the second magnetic coil unit 12 which faces away from the gap 14. The cabins 41 and 42 are preferably embodied large enough for people to remain inside. The first high frequency shielding cabin 41 comprises a first door 44, the second high frequency shielding cabin 42 can comprise a second door 45, in order if necessary to provide operating personnel with access from both sides.

The first cabin is connected to the first magnetic coil unit 11 in a high frequency sealed fashion. The second cabin 42 is connected to the second magnetic coil unit 12 in a high frequency sealed fashion. A tubular shielding unit 43 is disposed inside the first and second magnetic coil unit 11, 12 and bridges the gap 14 and connects the first magnetic coil unit 11 with the second magnetic coil unit 12 in a high frequency sealed fashion. The first and the second high frequency shielding cabins 41, 42 and the tubular high frequency shielding unit 43 can be made of copper sheeting and together with the two magnetic coil units 11, 12 form a Faraday cage.

### LIST OF REFERENCE CHARACTERS

1 Magnetic resonance imaging device

2 Linear accelerator/linac

3 Patient

4 High frequency shielding arrangement

11 First magnetic coil unit

12 Second magnetic coil unit

13 Longitudinal axis

14 Gap

21 X-ray radiation

41 First high frequency shielding cabin

42 Second high frequency shielding cabin

43 Tubular high frequency shielding unit

44 First door

45 Second door

1. A radiation therapy system, comprising:
a magnetic resonance imaging device comprising a first magnetic coil unit and a second magnetic coil unit, wherein the first magnetic coil unit and the second magnetic coil unit are distanced from each other by a gap and interact with each other to generate a magnetic field propagating along a longitudinal axis of the system;
a linear accelerator arranged at a radial distance from the longitudinal axis; and
a high frequency shielding arrangement comprising:
a first high frequency shielding cabin arranged adjacent to the first magnetic coil unit, and
a second high frequency shielding cabin arranged adjacent to the second magnetic coil unit, and
a tubular high frequency shielding unit arranged along the longitudinal axis that high frequency tightly connects the first high frequency shielding cabin to the second high frequency shielding cabin.

2. The radiation therapy system as claimed in claim 1, wherein the first and/or the second high frequency shielding cabin is large enough to remain a person inside.

3. The radiation therapy system as claimed in claim 1, wherein the first high frequency shielding cabin comprises a first door.

4. The radiation therapy system as claimed in claim, wherein the second high frequency shielding cabin comprises a second door.

5. The radiation therapy system as claimed in claim 1, wherein the tubular high frequency shielding unit is configured to penetrate energy-rich x-rays of the linear accelerator.

6. A radiation therapy system, comprising:
a magnetic resonance imaging device comprising a first magnetic coil unit and a second magnetic coil unit, wherein the first magnetic coil unit and the second magnetic coil unit are distanced from each other by a gap and interact with each other to generate a magnetic field propagating along a longitudinal axis of the system;
a linear accelerator arranged at a radial distance from the longitudinal axis; and
a high frequency shielding arrangement comprising:
a first high frequency shielding cabin arranged adjacent to the first magnetic coil unit, and
a second high frequency shielding cabin arranged adjacent to the second magnetic coil unit, and
a tubular high frequency shielding unit arranged along the longitudinal axis that high frequency tightly connects the first high frequency shielding cabin to the second high frequency shielding cabin, wherein the tubular high frequency shielding unit is high frequency sealed connected to the first magnetic coil unit and the second magnetic coil unit.

7. The radiation therapy system as claimed in claim 6, wherein the first and/or the second high frequency shielding cabin is large enough to remain a person inside.

8. The radiation therapy system as claimed in claim 6, wherein the first high frequency shielding cabin comprises a first door.

9. The radiation therapy system as claimed in claim 6, wherein the second high frequency shielding cabin comprises a second door.

10. The radiation therapy system as claimed in claim 6, wherein the tubular high frequency shielding unit is configured to penetrate energy-rich x-rays of the linear accelerator.
